(19) United States
(12) Patent Application Publication
(10) Pub. No.: US 2006/0241969 A1
(43) Pub. Date: Oct. 26, 2006

(54) Health cost avoidance system

(76) Inventors: Calvin Christian Wilhide, Lutherville, MD (US); Claire B. Rosse, Baltimore, MD (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 11/110,469

(22) Filed: Apr. 20, 2005

(51) Int. Cl. G06Q 10/00; A61B 5/00

(52) U.S. Cl. 705/2; 600/300

(57) ABSTRACT

Complications of various diseases have ascertainable direct costs and indirect costs. A method of projecting direct and indirect cost savings which may be realized from targeted interventions for achieving desired clinical outcomes which may result in the avoidance or mitigation of the complication is provided. Further, a comprehensive patient care management program is provided whereby the targeted intervention strategy of the present invention provides a list of behavioral influencers which assist in achieving desired clinical outcomes. From the achievement of the desired clinical outcomes direct and indirect cost savings from the avoidance or mitigation of a complication provides a projected overall cost savings which may be realized for an employer sponsored health plan.

### FIELD OF THE INVENTION

The present invention generally relates to the field of cost analysis for employer sponsored health plans, and particularly to a system for projecting direct and indirect health care cost savings which may be realized from specific mitigating interventions taken for a complication of a disease.

### BACKGROUND OF THE INVENTION

In the field of health care escalating costs are a common concern for both the consumer and provider alike. Numerous health care service/benefit programs, i.e., managed care, group health, and the like, have attempted to stem these rising costs by utilizing cost projection analysis techniques. Traditionally, disease management companies only provide employer sponsored health plans with considerations of hard savings realized when calculating the impact of their disease management programs. Thus, the disease management programs quantify the direct costs associated with a particular medical procedure, disease, and/or course of treatment. The currently employed cost projection techniques may only be providing a return on investment analysis, based on these direct costs. While hard savings are significant when measuring the effectiveness of a disease management program, they are only part of a larger picture. Unfortunately, the direct cost return on investment analysis provides a limited view of the total costs which may be associated with managing a disease. It is important to be able to quantify all cost savings, including the costs related to the avoidance and/or treatment of future complications which may arise during the progression of a disease.

Beyond the direct health care costs of managing a disease are the indirect costs, such as the costs associated with absenteeism, presentism, and/or disability. Presentism being that condition where the employee is at work but is not productive. The productivity in the case of presentism may vary from relatively small decreases in productivity to an absence of productivity. These indirect costs, which are commonly borne by employers who lose productivity due to absenteeism and/or presentism and the employers who have to pay for workers compensation due to disability, are often not accounted for by the current cost projection techniques. However, the indirect costs are not insignificant. For example, the American Association of Clinical Endocrinologist Diabetes Guide, Vol. 6, No. 1, January/February 2000, which is herein incorporated by reference in its entirety, published a report stating that the total indirect costs in 1997 for diabetes was calculated at fifty-four billion dollars ($54,000,000,000 USD). With the realization that this is only one disease among many which may have significant indirect costs associated with its progression and treatment, it is clear that a more comprehensive cost projection model/technique would benefit the field of disease management as part of an overall health care plan. The potential savings associated with improved health, effective preventative treatment and/or the avoidance of complications which may arise from the progression of diseases can be considerable.

Additionally, it is often the case that many of the complications associated with disease are mitigable to a varying extent by proper course of treatment and/or an effective disease management plan. Through such preventative action the benefits of mitigating intervention activities may be realized to the patient but also to the cost of providing health care under a health plan. Unfortunately, current cost projection modeling techniques may fail to identify these types of indirect cost savings in a quantifiable manner and therefore the cost savings projected may fail to provide an accurate account of the benefit of these programs.

Therefore, it would be desirable to provide a health care cost projection technique which is able to provide quantified cost savings for both direct and indirect costs attributed to a chronic disease. Further, a health care cost projection technique which is able to quantify savings on indirect costs associated with targeted mitigating intervention activities taken for the treatment of the progression of a disease is desirable for providing a more accurate reflection of the total cost savings which may be realized by an employer sponsored group.

### SUMMARY OF THE INVENTION

Accordingly, the present invention provides a method for projecting health care cost savings/avoidance based on the direct and indirect costs associated with the progression of a particular disease. By determining cost savings attributable to the attainment of desired clinical outcomes achieved by targeted mitigating interventions for a complication arising during the progression of the particular disease a more accurate cost savings/avoidance projection model is achieved. Therefore, the cost savings assumptions are applied to the successful achievement of clinical outcomes through targeted interventions and demonstrate the economic value of these clinical outcomes/targeted interventions as part of a disease management program in the context of direct medical expenses and indirect costs.

It is an object of the present invention to assist in providing a systematic and comprehensive approach to patient and/or disease management. Through use of the present invention, patients may be provided with an individualized care plan which may influence behavior, such behavior influences being directed towards increased health for the patient and cost savings/avoidance for the health care plan provider by proper disease management which accounts for the direct and indirect costs associated with the progression of various diseases. Using a prevalence-based focus on disease-specific behaviors that may have the most significant impact on improving individual health the present invention relates the achievement of desired clinical outcomes/targeted interventions to the avoidance of complications, such complications are based on the progression of a disease over time. By focusing on determining what interventions have the most impact in improving a patient's health, future costs of complications of the disease may be mitigated/avoided. The focused intervention strategy of the present invention is based on a hierarchy of targeted interventions which achieve a desired clinical outcome and have the most impact upon the progression of a disease, particularly the development of complications associated with the disease.

In a first aspect of the present invention, a method of projecting health care cost savings for a complication associated with a disease includes determining an indirect cost associated with the complication. Once the indirect cost is determined then a mitigating factor associated with the complication is determined. The mitigating factor is the percent mitigable of the complication. Mitigation of the complication occurs through the use of targeted interventions that achieve desired clinical outcomes, which may assist in reducing the risk of developing a complication and/or reducing the amount of necessary treatment for the complication. Utilizing the determined indirect cost and the mitigating factor a projected indirect cost savings is calculated.

In a second aspect of the present invention, a method of projecting health care cost savings for a complication associated with a disease includes determining indirect and direct costs associated with the complication. A mitigating factor is then determined and in combination with the direct and indirect costs a total cost savings projection is calculated. It is an object of the present invention to provide cost savings projections for both direct costs and indirect costs which may be utilized independently.

It is a further object of the present invention to provide the cost savings projection method for various complications associated with the progression of various diseases. By providing and using the indirect costs associated with a complication of a disease, the present invention provides a significant advantage over the prior art. The cost savings projection of the present invention goes beyond the limited return on investment analysis of direct costs typified by current health plan cost projection modeling techniques and gives quantifiable (return on investment) information on direct and indirect costs associated with such things as absenteeism, disability, and presentism. These indirect costs may amount to billions of dollars per year lost to providers and consumers of health plans. With the cost savings projection enabled by the present invention, the employers and/or consumers are provided a more detailed and accurate account of all the possible benefits, related to direct and indirect costs, of various alternative care strategies before having to implement them.

In a third aspect of the present invention, a computer readable set of instructions (software program) allowing for the projection of health care cost savings for a complication associated with a disease, is provided. A first instruction determines a direct cost savings associated with the complication and a second instruction determines an indirect cost savings associated with the complication. Using these determined values a third instruction calculates a projected total cost savings which may be associated with the complication.

It is an object of the present invention to present a method of non-claims based calculation of savings which applies cost savings assumptions to the successful achievement of clinical outcomes/targeted interventions. This allows the present invention to provide program benefit information to those employer sponsored health plans which do not have credible population pool size.

It is to be understood that both the foregoing general description and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1 is a block diagram illustrating a method for projecting health care cost savings for indirect costs in accordance with an exemplary embodiment of the present invention;

FIG. 2 is a block diagram illustrating a method for projecting health care cost savings for total costs, including direct and indirect costs, in accordance with an exemplary embodiment of the present invention;

FIG. 3 is a block diagram illustrating a computer readable set of instructions for projecting health care cost savings for a disease; and

FIG. 4 is an illustration representative of the development of an individualized patient care/disease management program in accordance with an exemplary embodiment of the present invention.

### DETAILED DESCRIPTION OF THE INVENTION

Reference will now be made in detail to the presently preferred embodiments of the invention, examples of which are illustrated in the accompanying drawings.

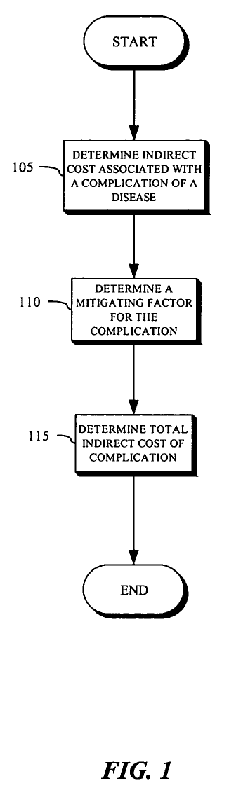

Referring generally now to FIG. 1, a method projecting health care cost savings for indirect costs associated with a disease, is shown. In a first step 105, an indirect cost for a complication associated with the progression of a disease is determined. An indirect cost may be absenteeism, presentism, and/or a disability, associated with the disease and a complication arising from the progression of the disease. For example, using diabetes as a disease model, a complication may include a kidney transplant, end-stage renal disease, amputations/gangrene, stroke, ketoacidosis, diabetic nephropathy, infections/advanced infections, diabetic neuropathy, diabetic retinopathy, coronary artery disease (CAD), chronic (congestive) heart failure (CHF), and the like. As will be described in detail below the indirect cost is the cost as a percentage of per capita charges associated with the particular complication. Once the indirect cost for the complication of the disease is determined, then in step 110 a mitigating factor is determined. The mitigating factor is the percent mitigable of the indirect cost of the disease. The percent mitigable is based on the attainment of a specific clinical outcome through targeted intervention which results in the avoidance and/or mitigation of a complication of the disease and is determined by analysis (meta-analysis) of clinical trials. The targeted intervention-may include medication management, diet, exercise, periodicity (doctor visits) management, smoking cessation or various other interventions as contemplated by those of ordinary skill in the art. The desired clinical outcome may be to achieve a particular blood sugar (Hb1Ac) level, blood pressure, cholesterol level, and the like.

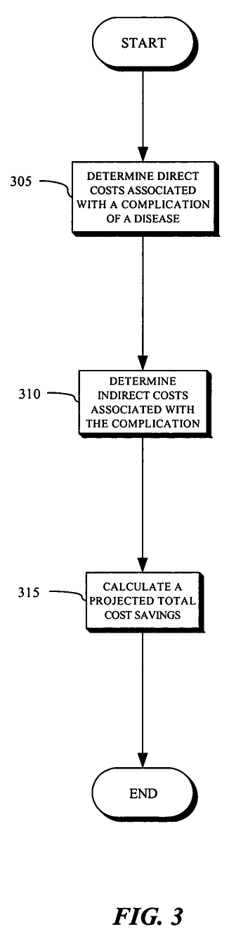

From the diabetic model it is known that the complication of ketoacidosis associated with diabetes is mitigable by fifty percent (50%). Therefore, the mitigating factor for ketoacidosis is 0.50. In step 115 the total indirect cost savings may be determined utilizing the indirect cost and the mitigating factor. This determination is made by multiplying the indirect cost by the mitigating factor to achieve the total indirect cost savings projection. From this a disease management program or the like is enabled to identify the indirect cost savings which may be realized. It is contemplated that the method of the present invention may be executed upon a computing system as a computer readable set of instructions, as is described below in detail in reference to FIG. 3.

Included below is an example of the determination of the cost savings which may be projected by the present invention for a disease. The disease of this example is diabetes.

Using the reported per capita healthcare cost for a diabetic in 1997 ($10,071), See American Association of Clinical Endocrinologist Diabetes Guide, Vol. 6, No. 1, January/February 2000, and applying the medical cost inflation rate, the 2003 per capita cost per diabetic is projected to be $18,052.37. Prevalence rates for the most common and costly complications associated with diabetes were determined. The distribution of cost associated with these complications was calculated from the projected per capita cost for diabetes by dividing each complication prevalence rate by the sum of the complication prevalence rates. These values were termed the adjusted prevalence rates. Met-analysis of clinical trials targeting specific interventions/clinical outcomes provided the mitigable impact that a successful intervention, resulting in the attainment of a desired clinical outcome, confers. The per capita cost associated with each complication was calculated by multiplying the adjusted prevalence rate for the complication and the 2003 projected per capita cost for diabetes. These values are termed cost as percentage of per capita charges. Next, the percentage mitigable impact for each complication was multiplied by the cost as percentage of per capita charges. This yielded the projected direct cost savings per complication associated with successful specific interventions (Table 1).

TABLE 1

| | | Adjusted | | Cost As % Of | | Cost |
|---|---|---|---|---|---|---|
| Diabetic | Prevalence | Prevalence | Cost To Treat | Per Capita | Percent | Savings/ |
| Complication | Rate | Rate | For 2003 | Charges | Mitigable | Diabetic |
| Kidney Transplant | 0.30% | 0.13% | $100,000 | $422.63 | 0% | $0.00 |
| End-Stage Renal | 0.63% | 0.26% | $180,000 | $47.14 | 0% | $0.00 |
| Disease | | | | | | |
| Amputations/ | 3.50% | 1.46% | $44,160 | $264.01 | 0% | $0.00 |
| Gangrene | | | | | | |
| Stroke | 1.00% | 0.42% | $54,203.38 | $75.43 | 45% | $33.94 |
| Ketoacidosis | 8.90% | 3.72% | $16,000 | $671.33 | 50% | $335.67 |
| Diabetic | 16.00% | 6.69% | $37,156.69 | $1,206.89 | 50% | $603.44 |
| Nephropathy | | | | | | |
| Infections/Advanced | 25.00% | 10.45% | $20,805 | $1,885.76 | 50% | $942.88 |
| Infections | | | | | | |
| Diabetic Neuropathy | 50.00% | 20.89% | $20,100 | $3,771.52 | 60% | $2,262.91 |
| Diabetic | 59.00% | 24.65% | $16,264.12 | $4,450.39 | 65% | $2,892.75 |
| Retinopathy | | | | | | |
| CAD (CHF) | 75.00% | 31.34% | $28,036.29 | $5,657.28 | 24% | $1,357.75 |
| Total | 239.33% | 100.00% | | $18,052.37 | | $8,429.34 |

The indirect cost per diabetic was calculated using published data on the total indirect cost of diabetes in 1997 ($54 Billion) and the estimated number of diabetics in 1997 (267 Million people×6.0 percent incidence rate=16 Million diabetics). From these figures, it may be estimated that the indirect cost per diabetic in 1997 was $3,375.00. This value was adjusted to a projected 2003 value ($3,893.60) using published inflation rates for corresponding years. The same set of calculations as described above were used to generate indirect cost savings per diabetic complication (Table 2).

TABLE 2

| | | Adjusted | | Cost As % Of | | Cost |
|---|---|---|---|---|---|---|
| Diabetic | Prevalence | Prevalence | Cost To Treat | Per Capita | Percent | Savings/ |
| Complication | Rate | Rate | For 2003 | Charges | Mitigable | Diabetic |
| Kidney Transplant | 0.30% | 0.13% | $100,000 | $4.88 | 0% | $0.00 |
| End-Stage Renal | 0.63% | 0.26% | $180,000 | $10.17 | 0% | $0.00 |
| Disease | | | | | | |
| Amputations/ | 3.50% | 1.46% | $44,160 | $56.94 | 0% | $0.00 |
| Gangrene | | | | | | |
| Stroke | 1.00% | 0.42% | $54,203.38 | $16.27 | 45% | $7.32 |
| Ketoacidosis | 8.90% | 3.72% | $16,000 | $144.79 | 50% | $72.40 |
| Diabetic | 16.00% | 6.69% | $37,156.69 | $260.31 | 50% | $130.15 |
| Nephropathy | | | | | | |
| Infections/Advanced | 25.00% | 10.45% | $20,805 | $406.73 | 50% | $203.36 |
| Infections | | | | | | |
| Diabetic Neuropathy | 50.00% | 20.89% | $20,100 | $813.45 | 60% | $488.07 |
| Diabetic | 59.00% | 24.65% | $16,264.12 | $959.88 | 65% | $623.92 |
| Retinopathy | | | | | | |
| CAD (CHF) | 75.00% | 31.34% | $28,036.29 | $1,220.18 | 24% | $292.84 |
| Total | 239.33% | 100.00% | | $3,893.60 | | $1,818.07 |

The sum of the direct and indirect cost savings calculated for the present invention is grouped according to the intervention, which mitigated the specific diabetic complication. Using the American Diabetes Association's 2003 guidelines (clinical trials), interventions with a grade of A or B (indicating level of evidence) were used as the criteria for assigning a diabetic complication to a clinical outcome. This value represents the cost savings associated with attaining a clinical outcome in that category (Table 3).

TABLE 3

| | | | Direct Cost | Indirect Cost | Total Cost |
|---|---|---|---|---|---|
| | Attainment | Diabetic | Savings Associated | Savings Associated | Savings For |
| | of Clinical | Complication | With Mitigated | With Mitigated | Attaining |
| Value | Indicator | Impacted | Complication | Complication | Goal/Diabetic |
| 1 | HbA1c level | | | | |
| | | Ketoacidosis | $335.67 | $72.40 | $408.07 |
| | | Diabetic | $2,262.91 | $488.07 | $2,750.98 |
| | | Neuropathy | | | |
| | | Infections/ | $942.88 | $203.36 | $1,146.24 |
| | | Advanced | | | |
| | | Infections | | | |
| | Sub-total | | | | $4,305.29 |
| 2 | HbA1c | | | | |
| | Level and | | | | |
| | BP | | | | |
| | | Diabetic | $2,892.75 | $623.92 | $3,516.67 |
| | | Retinopathy | | | |
| | | Diabetic | $603.44 | $130.15 | $733.59 |
| | | Nephropathy | | | |
| | Sub-total | | | | $4,250.26 |
| | Cumulative | | | | $8,555.55 |
| | Total | | | | |
| 3 | HbA1c | | | | |
| | Level, | | | | |
| | Blood | | | | |
| | Presssure, | | | | |
| | Cholesterol | | | | |
| | | Stroke | $33.94 | $7.32 | $41.26 |
| | | CAD (CHF) | $1,357.75 | $292.84 | $1,650.59 |
| | Sub-total | | | | $1,691.85 |
| | Grand Total | | | | $10,247.40 |

While the method for cost savings, both direct and indirect costs, related to diabetes and the effects of specific mitigating interventions upon certain complications associated with diabetes has been disclosed above, it is to be understood that the present invention may be applied to various diseases/medical conditions providing various disease management programs with a more comprehensive cost savings projection technique.

In the alternative the method for cost savings projection is applied to costs associated with back pain. In the year 2000 it was determined that approximately 5.83% of the adult population of the United States of America has a back problem in any given year. The USA population in 2000 was 281,421,906, therefore the number of adults being treated for back problems that year was approximately 12, 148,046. The total overall estimated costs for back problems in the year 2000 was $100,000,000,000 with direct costs (medical disability, lost work) accounting for approximately $50,00,000,000 and indirect costs (lost productivity) accounting for approximately $50,000,000,000. The method of the present invention may be employed in a similar manner for back problems as it was for diabetes, as shown in Table 4.

TABLE 4

| | | Adjusted | | Cost As % Of | | Cost |
|---|---|---|---|---|---|---|
| | Prevalence | Prevalence | Cost To Treat | Per Capita | Percent | Savings/ |
| Back Problems | Rate | Rate | For 2003 | Charges | Mitigable | Back Pain |
| Disk Degradation, | 90% | 90% | $0-$10,000 | $3,120.19 | 50% | $1,560.10 |
| Ruptured Disks, | | | | | | |
| Pinched Nerves, | | | | | | |
| Muscle Strain | | | | | | |
| Spinal tumors | 0.70% | 0.70% | $30,000 | $24.27 | 0% | $0.00 |
| Infectious | 0.10% | 0.10% | $7,000 | $3.47 | 0% | $0.00 |
| Major Trauma | 4.00% | 4.00% | $50,000 | $138.68 | 0% | $0.00 |
| Congenital | 3.00% | 3.00% | $1,000-$20,000 | $104.01 | 0% | $0.00 |
| Abnormalities | | | | | | |
| Other (Cauda | 2.20% | 2.20% | $30,000 | $76.27 | 0% | $0.00 |
| Equina Syndrome) | | | | | | |
| Total | 100% | 100.00% | | $3,466.88 | | $1,560.10 |

From this it was estimated that the total cost for Back problems per patient per year without interventions was $3,466.88 in 2003. If we subtract from this the determined cost savings of $1,560.10 we can calculate that the total cost per patient per year after specific mitigating interventions are taken is equal to $1,906.78. From this it may be determined that the projected cost savings based on projected 2003 goal attainment (achieving specific clinical outcomes) was $780.05. Thus, the total cost adjusted for projected 2003 goal attainment is $2,686.83 a reduction of $801.83 which equates to a realized reduction in costs for back problems of approximately 23%.

The method of cost savings projection may also be applied to asthma, as shown below in Table 5. In the year 1998 it was determined that approximately 6.00% of the adult population of the United States of America had asthma. The USA population in 1998 was 270,311,756, therefore the number of adults being treated for asthma events that year was approximately 16,218,705. The total overall estimated costs for asthma problems in the year 1998 was approximately $12,670,000,000 with direct costs (hospital inpatient, ER, hospital outpatient, physician inpatient, office visit, prescription) accounting for approximately $7,365,000,000 and indirect costs (lost productivity-presentism: lost school days, lost work men, lost work women, house keeping, mortality) accounting for approximately $5,305,000,000.

TABLE 5

| | | Adjusted | Cost As % Of | | Cost |
|---|---|---|---|---|---|
| | Prevalence | Prevalence | Per Capita | Percent | Savings/ |
| Asthma Event | Rate | Rate | Charges | Mitigable | Back Pain |
| Hospital Inpatient | 29% | 29% | $1,678.72 | 60% | $1,007.23 |
| Emergency | 7.50% | 7.50% | $434.15 | 60% | $260.49 |
| Department | | | | | |
| Hospital Outpatient | 10% | 10% | $578.87 | 60% | $347.32 |
| Physician Inpatient | 1.50% | 1.50% | $86.83 | 60% | $52.10 |
| Physician Office | 10% | 10% | $578.87 | 0% | $0.00 |
| Prescriptions | 42% | 42% | $2,431.25 | 0% | $0.00 |
| Total | 100% | 100.00% | $5,788.69 | | $1,667.14 |

It was estimated that the total cost for asthma problems per patient per year without interventions was $5,788.69 in 2003. If we subtract from this the determined cost savings of $1,667.17 we can calculate that the total cost per patient per year after specific mitigating interventions are taken is equal to $4,121.55. A break down analysis of the determined cost savings from Table 5 is provided below in Table 6. The total cost savings for achievement of targeted interventions resulting in the attainment of desired clinical indicators is broken down by the affect the interventions have upon four medical events, those being (1) Hospital Inpatient, (2) Emergency Department, (3) Hospital Outpatient and (4) Physician Inpatient. Table 6.

TABLE 6

| | | | Total Cost |
|---|---|---|---|
| | Attainment of Clinical | Medical Event | Savings For Attaining |
| Value | Indicator | Affected | Goal/Asthmatic |
| 1 | Asthmatic, controlled | | |
| | | Hospital Inpatient | $503.62 |
| | | Emergency | $130.25 |
| | | Department | |
| | | Hospital Outpatient | $173.66 |
| | | Physician Inpatient | $26.05 |
| | Sub-total | | $833.57 |
| Cumulative | | | |
| Total | | | |
| 2 | Compliance to Asthma | | |
| | Medication | | |
| | | Hospital Inpatient | $251.81 |
| | | Emergency | $65.12 |
| | | Department | |
| | | Hospital Outpatient | $86.83 |
| | | Physician Inpatient | $13.02 |
| | Sub-total | | $416.79 |
| Cumulative | | | |
| Total | | | |
| 3 | Compliance With Peak Flow | | |
| | | Hospital Inpatient | $251.81 |
| | | Emergency | $65.12 |
| | | Department | |
| | | Hospital Outpatient | $86.83 |
| | | Physician Inpatient | $13.02 |
| | Sub-total | | $416.79 |
| Cumulative | | | $1,667.14 |
| Total | | | |

From the results shown in Table 5 and 6 it was determined that the projected cost savings based on projected 2003 goal attainment (achieving specific clinical outcomes) was $1,000.28. Thus, the total cost adjusted for projected 2003 goal attainment is $5,121.83 a reduction of $666.86 which equates to a realized reduction in costs for asthma problems of approximately 11.5%.

The method of cost savings projection may also be applied to coronary artery disease (CAD), as shown in Table 7. Based on figures for calendar year 2003 and utilizing the same principles illustrated and described previously, the direct and indirect cost savings associated with the mitigation of a complication by attainment of a clinical indicator through targeted intervention may be determined.

TABLE 7

| | | | Direct Cost savings | Indirect Cost savings | Total cost |
|---|---|---|---|---|---|
| | | | associated with | associated with | saving for |
| | Attainment of | CAD complication | mitigated | mitigated | Attaining |
| Value | clinical indicator | impacted | complication | complication | Goal/CAD |
| 1 | BP | | | | |
| | | Angina | $435.09 | $17.81 | $452.90 |
| | | Dysrhythmia | $146.43 | $5.99 | $152.42 |
| | | Stroke | $527.13 | $21.58 | $548.71 |
| | | MI | $502.03 | $20.55 | $522.58 |
| | | Revascularization | $836.72 | $34.25 | $870.97 |
| | | CHF | $326.32 | $13.36 | $339.68 |
| | Sub-total | | | | $2,887.26 |
| 2 | Cholesterol | | | | |
| | | Angina | $435.09 | $17.81 | $452.90 |
| | | Dysrhythmia | $146.43 | $5.99 | $152.42 |
| | | Stroke | $527.13 | $21.58 | $548.71 |
| | | MI | $502.03 | $20.55 | $522.58 |
| | | Revascularization | $836.72 | $34.25 | $870.97 |
| | | CHF | $326.32 | $13.36 | $339.68 |
| | Sub-total | | | | $2,887.26 |
| 3 | Medication | | | | |
| | Management | | | | |
| | | Post MI | $3,703.70 | $151.62 | $3,855.32 |
| | Sub-total | | | | $3,855.32 |
| | Grand Total | | | | $9,629.84 |

Therefore, for 2003 the projected cost savings, determined through application of the method of the present invention, was $9,629.84. This projected cost savings reflects both direct cost and indirect cost savings which may result through the attainment of desired clinical outcomes through targeted intervention.

Table 8 shows the application of the cost savings projection method of the present invention for chronic (congestive) heart failure (CHF). From this it may be determined that a total cost savings of $8,280.48 may be realized by health plans and/or consumers of health plans.

TABLE 8

| | | | Direct Cost savings | Indirect Cost savings | |
|---|---|---|---|---|---|
| | Attainment of | | associated with | associated with | Total cost savings |
| | clinical | CAD complication | mitigated | mitigated | for Attaining |
| Value | indicator | impacted | complication | complication | Goal/CAD-CHF |
| 1 | BP | | | | |
| | | Angina | $435.09 | $17.81 | $452.90 |
| | | Dysrhythmia | $146.43 | $5.99 | $152.42 |
| | | Stroke | $527.13 | $21.58 | $548.71 |
| | | MI | $502.03 | $20.55 | $522.58 |
| | | Revascularization | $836.72 | $34.25 | $870.97 |
| | | CHF | $326.32 | $13.36 | $339.68 |
| | Sub-total | | | | $2,887.26 |
| 2 | Cholesterol | | | | |
| | | Angina | $435.09 | $17.81 | $452.90 |
| | | Dysrhythmia | $146.43 | $5.99 | $152.42 |
| | | Stroke | $527.13 | $21.58 | $548.71 |
| | | MI | $502.03 | $20.55 | $522.58 |
| | | Revascularization | $836.72 | $34.25 | $870.97 |
| | | CHF | $326.32 | $13.36 | $339.68 |
| | Sub-total | | | | $2,887.26 |
| 3 | Medication | | | | |
| | Management | | | | |
| | | CHF | $2,407.41 | $98.55 | $2,505.96 |
| | Sub-total | | | | $2,505.96 |
| | Grand Total | | | | $8,280.48 |

Thus, the per capita cost savings which may be realized per complication, including both the direct and indirect costs, is projected by the method of the present invention. In the above examples, a disease management program or any health plan which implements the goals of attaining the clinical indicators shown is able to project the per complication return on investment into the targeted interventions for achieving these goals.

It is contemplated that the method of cost savings projection for complications associated with the progression of disease, may be applied to various diseases. Having described the cost savings projection method using numerous specific examples, it is appreciated by those of ordinary skill in the art that the method is not limited to the diseases of the examples provided but may be applied to all known diseases where the direct and indirect costs of complications arising from the progression of the disease are identified.

The present invention provides a method for projecting health care cost savings for direct costs of a complication associated with a disease. In a first step a direct cost associated with the complication arising from the progression of a disease is determined. The complications are identified in Table 1 above, and are similar to those described above in reference to indirect costs. For example, the complications may include a kidney transplant, end-stage renal disease, amputations/gangrene, stroke, ketoacidosis, diabetic nephropathy, infections/advanced infections, diabetic neuropathy, diabetic retinopathy, coronary artery disease, chronic (congestive) heart failure, and the like.

Once the direct cost for the disease is determined, then in a second step a mitigating factor is determined. The mitigating factor is the percent mitigable of the determined direct cost of the disease. The percent mitigable is based on the attainment of a specific clinical outcome by targeted intervention to avoid and/or treat the complication. As previously identified, the specific mitigating interventions may include medication management, diet, exercise, periodicity (doctor visits) management, and or smoking cessation. The desired clinical outcome may be to achieve a particular Hb1Ac level, blood pressure, cholesterol level, and the like. Using the diabetic model, the complication of ketoacidosis associated with diabetes is mitigable by fifty percent (50%), thereby, having a mitigating factor of 0.5.

In a third step the total direct cost savings may be determined utilizing the direct cost and the mitigating factor. This determination is made by multiplying the direct cost by the mitigating factor to achieve the total direct cost savings projection, from this the present invention allows a disease management program to identify the direct cost savings which may be realized by specific interventions.

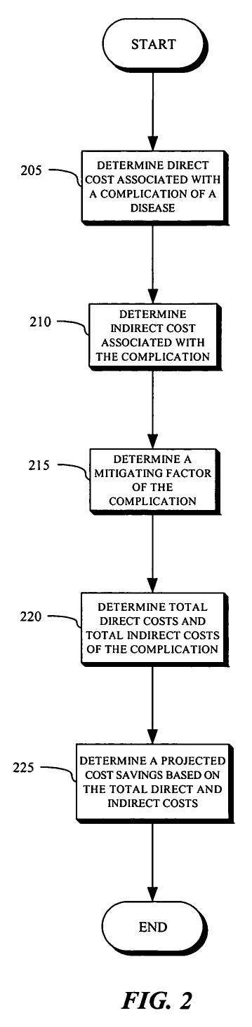

Referring now to FIG. 2, a method for projecting total health care cost savings based on direct and indirect health care costs of a complication developing from the progression of a disease is shown. It is to be understood that the method of the current embodiment is described using the above described diabetic model and tables, but that the method may be applied to various diseases/medical conditions and their associated complications. In a first step 205 a direct cost of a complication associated with a disease is determined. In a second step 210 an indirect cost of the complication associated with a disease is determined. In a third step 215 a mitigating factor, which is the percent mitigable of the determined direct cost and indirect cost, is determined. The direct cost and indirect cost associated with a disease and the mitigating factor may be determined in a manner similar to that for indirect and direct costs, described previously. In the current embodiment, the mitigating factor is equivalent for both the indirect and direct costs. It is contemplated that the mitigating factor may be different for the indirect cost as compared to the direct cost. Such a difference in the mitigating factor may provide increases or decreases in projected cost savings which may be realized without departing from the scope and spirit of the present invention. Once the direct costs, indirect costs, and mitigating factor have been calculated, then the total direct costs and indirect costs are determined in step 220. The total direct and indirect costs are determined in a manner similar to that shown and described above in Tables 1 and 2.

As shown in Table 3 above, the direct and indirect costs which were individually associated with a particular complication in Tables 1 and 2, respectively, are cumulatively associated with the particular complication. Therefore, in step 225 a projected cost savings based on targeted intervention of a particular complication is determined based on both the direct and indirect costs associated with that complication.

It is contemplated that the present invention may be implemented and performed as an executable computer program. In a preferred embodiment, the executable computer program is a software program that includes a set of computer readable instructions which may be executed upon a computing system (i.e., computer, PC, laptop). Referring now to FIG. 3, a block diagram representation of a computer readable set of instructions (software program) for projecting health care cost savings for execution upon a computing system, is shown. A first instruction 305 determines a projected direct cost savings for a complication associated with a disease. It is to be understood that the complication associated with the disease may be any one of the complications previously described in association with the various diseases. Further, the present invention contemplates the calculation of projected cost savings for more than one complication occurring in association with one or more diseases. Therefore, the present invention is capable of providing a cost savings projection analysis for multiple complications associated with one or more diseases.

The direct cost for treatment and the prevalence rate for each complication is determined and used to project the per capita cost of treatment. Further, for each complication a mitigating factor (percent mitigable) is determined. It is contemplated that the mitigating factor may range from zero percent mitigable to one hundred percent mitigable. Then the program multiplies the per capita cost of treatment by the mitigating factor for each complication to arrive at a direct cost savings. The direct cost savings projection is a projected direct cost savings per complication associated with successful targeted interventions which attain a desired clinical outcome.

As mentioned previously, the targeted interventions may be associated in a hierarchical manner to a desired clinical outcome to assist in avoiding/mitigating the specific complication. For example, under the diabetes model, the clinical outcome of achieving a specific blood-sugar level (HbA1c level) has the following hierarchy of interventions: (1) Medication Management, (2) Diet, (3) Exercise, (4) Periodicity and (5) Smoking Cessation. This hierarchy is based on the efficacy of the intervention in the achievement of the specific clinical outcome. It is contemplated that the listing of interventions may be organized using different principles. Further, the interventions may be used for achieving various alternative clinical outcomes, such as achieving a specified blood pressure, cholesterol level, and the like. The achievement of the various clinical outcomes may occur independently from or in combination with one another. The order of the hierarchies for the alternative clinical outcomes may be similar or vary from one another. For example, the clinical outcome of achieving a specified blood pressure and cholesterol level, under the diabetes model, both include the following hierarchy of interventions: (1) Medication Management, (2) Exercise, (3) Diet, (4) Periodicity and (5) Smoking Cessation.

It is to be understood that the clinical outcomes identified above are typical for a diabetes patient but in no way is intended to exclude other desired clinical outcomes for various other diseases or for diabetes. The desired cholesterol level clinical outcome may encompass all forms of cholesterol, in particular the high density lipoprotein (HDL) and low density lipoprotein (LDL) forms. It is also contemplated that the interventions, such as periodicity may be read to encompass several factors, such as HbA1c testing two to four times a year, annual foot exam, annual urine test for protein, annual dilated retinal eye exam, annual HDL, annual LDL, compliance with medication regiment, daily glucose monitoring, understanding of disease, sick day plan, blood pressure monitoring at every physician-visit, and the like.

A second instruction 310 directs the determination of indirect costs for the complication, in accordance with the principles previously described. The direct and indirect cost for treatment and the prevalence rate for each complication is determined and used to project the direct and indirect per capita cost of treatment. Further, for each complication a mitigating factor (percent mitigable) is determined. The mitigating factor may be the same for both the direct and indirect costs for the complication or each of the costs may have a unique mitigating factor associated with it. Then the program independently multiplies both the direct and indirect per capita cost of treatment by the mitigating factor for each complication to arrive at an indirect cost savings. The projected indirect cost savings are per complication and associated with successful specific interventions which attain a desired clinical outcome.

Using the direct and indirect cost savings, a third instruction 315, calculates a projected total cost savings. The projected total cost savings is based on the successful achievement of a desired clinical outcome by a specific intervention which impacts the complication. As stated previously, the impact upon the complication may range from avoidance of the complication, to reducing the risk of the patient being afflicted with complication, to decreasing the amount of necessary treatment for a patient afflicted with the complication. Thus, the computer readable set of instructions enables a computing system to provide a cost savings projection based on successful achievement of clinical outcomes by targeted interventions.

The computer readable set of instructions of the present invention may include an instruction allowing for the identification of the specific disease upon which the cost savings projections are made. For example, the program may allow for the input of the disease/medical condition directly, such as entry via a keyboard of the computing system of the disease/medical condition name, such as diabetes, asthma, hypertension, coronary artery disease, back pain, and the like. Alternatively, the present invention may allow for the entry of various symptoms. The various symptoms once entered may be analyzed and a diagnosis made by the method of the present invention as to what particular disease forms the basis for the cost savings projection. For example, the entry of symptoms, such as low insulin level and high blood sugar level, may allow for the present invention to diagnose that diabetes is the disease upon which its projections shall be formulated. In the current embodiment, a list of diseases is made available through storage of information within the software program. It is contemplated that various diseases/medical conditions may be utilized by the present invention from various locations. For instance, a listing of various diseases/medical conditions may be from a secondary computer readable storage device (i.e., diskette, CD-ROM, DVD, and the like), downloaded from the Internet and/or available from the computing system upon which the method is being executed. It is further contemplated that the list of disease/medical conditions may be updated to include new information or delete existing information without departing from the scope and spirit of the present invention. Further, the disease/medical condition information may be supplemented and/or replaced by the information being provided from a secondary source.

After the identification of the disease/medical condition, another instruction may command the computing system to construct a list of various complications associated with the identified disease/medical condition. For a patient with diabetes a list of complications may be similar to those shown above in Tables 1 and 2, including a kidney transplant, end-stage renal disease, amputations/gangrene, stroke, ketoacidosis, diabetic nephropathy, infections/advanced infections, diabetic neuropathy, diabetic retinopathy, coronary artery disease, and the like. The list of information may be stored within the software program itself, provided in a separate computer readable unit, downloaded from the Internet and/or stored within the computing system. It is to be understood that the listing of information associated with a particular disease/medical condition may be updated to include new information or delete existing information without departing from the scope and spirit of the present invention. For example, a computer readable storage media (i.e., diskette, CD-ROM, and the like) may include listings of information relevant to particular diseases/medical conditions which, when downloaded either supplements and/or replaces the existing information associated with the disease/medical condition.

In an alternative embodiment, the computer readable set of instructions (software program) may implement either a projected direct cost savings analysis or indirect cost savings analysis independently of one another. Therefore, the present invention may provide a cost savings projection based on either direct or indirect costs alone. Further, the program may provide an indication of various targeted interventions associated with the attainment of a desired clinical outcome for combating the complication. For instance, the computer readable set of instructions may include a database listing various targeted interventions (i.e., medication management, diet, exercise, periodicity, smoking cessation, and the like) associated in a hierarchical manner for the achievement of a desired clinical outcome. These interventions may be presented to the user, such as through use of a display screen of the computing system. It is contemplated that various techniques may be employed to provide this information to the user, such as through use of a print-out, letter, and the like.

In a preferred embodiment, the computer readable set of instructions of the present invention are contained upon an independent storage media, i.e., diskette, CD-ROM, and the like. Thus, the present invention may be implemented in a modular fashion, installing, replacing or supplementing various other programs. However, it is contemplated that the computer readable set of instructions provided by the present invention may be implemented within various other software programs. For example, a health plan may be contained and administered upon a digital framework. Through use of one or more various computing systems operators/administrators of the health plan may access this digital framework. Within the digital framework of the health plan may be included a disease management program. The disease management program may provide information on various diseases and associated complications. The present invention may be downloaded into or written onto the health plan and either replace or supplement the existing disease management program. The present invention may include an instruction to delete existing disease management information or an instruction which allows the present invention to incorporate the use of the existing information and place the instructions of the present invention within the digital framework of the existing disease management program.

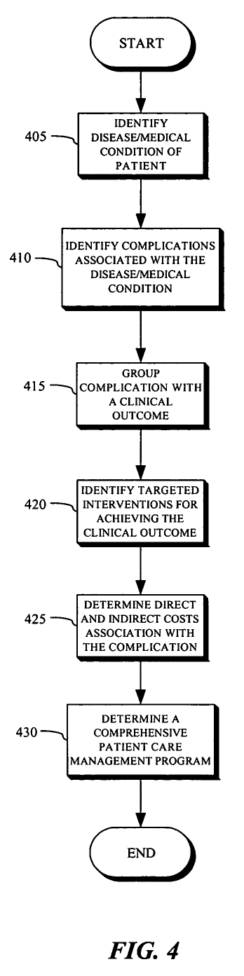

Referring now to FIG. 4, a method for providing individualized patient care/disease management programs using the projected cost savings technique of the present invention is shown. Patients are provided with an individualized care plan which influences behavior patterns of the patient. These behavior influences focus on increasing health for the patient and cost savings/avoidance for the health care plan provider by proper disease management which accounts for the direct and indirect costs associated with the progression of various diseases. Using the prevalence-based focus of the present invention, disease-specific behaviors (targeted interventions for achieving desired clinical outcomes) that may have the most significant impact on improving individual health are implemented within the context of the disease management program. Thus, the present invention relates the achievement of desired clinical outcomes/targeted interventions to the avoidance of complications, which may develop during the progression of a disease over time. By focusing on determining what interventions have the most impact in improving a patient's health, costs for complications of the disease may be mitigated/avoided.

In a first step 405 of the method, a patient's disease/medical condition is identified. This identification process is ultimately the result of a medical diagnosis, but may be provided from various sources, such as a doctor, the patient, other people with knowledge of the diagnosis, and the like. The disease/medical condition may be one of several, which may include but is not limited to diabetes, asthma, back problems, coronary artery disease, chronic heart failure, and other conditions as may be contemplated by those of ordinary skill in the art. After identification of the disease/condition, in step 410, a complication(s) associated with the progression of the disease/condition are identified. These complications may be identified from various sources, such as medical texts, treatises, journals, and the like. It is not necessary that the patient be experiencing the complication(s) at the time of identification of the complication(s) associated with the progression of the disease. Further, the present invention contemplates that a complication(s) may be identified at any time and included within the patient care management program of the present invention. The complications may be grouped in various manners, such as in a decreasing/increasing order of risk for development during disease progression, decreasing/increasing order of mortality, or various other manners as contemplated by those of skill in the art.

Clinical outcomes are identified and grouped with the complication(s), the clinical outcomes are identified by the impact upon the complication it has for the patient, in step 415. The impact, mitigating effect or avoidance effect, of the clinical outcome upon the complication is used to identify one or more desired clinical outcomes which may have an increased impact upon the complication and progression of the disease versus other identified clinical outcomes. With the desired clinical outcome(s) identified, one or more targeted interventions are identified which assist in the achievement of the clinical outcomes in step 420. The targeted interventions may be further categorized by the ability of the intervention to assist in promoting the achievement of the desired clinical outcome. Thus, the targeted intervention(s) may be arranged in a hierarchical manner whereby they are provided in a decreasing order of effectiveness in assisting the achievement of the clinical outcome. It is contemplated that they may be provided in an increasing order of effectiveness or various other arrangements without departing from the scope and spirit of the present invention. Using the identified complications and the projected cost savings technique of the present invention, the direct and indirect projected costs savings associated with each complication is determined in step 425. The determination of the direct and indirect projected costs savings is achieved through use of the method of the present invention described previously. The patient care management program may provide the projected cost savings information as the cumulative total from both the direct and indirect costs. Alternatively, the program may provide the projected costs savings information with direct costs separate from indirect costs.

With the disease, complications and their projected cost savings, and the targeted interventions for achieving desired clinical results identified, a comprehensive, individualized disease management program is determined in step 430. For example, using the information from Tables 1 and 2 above and knowing a person has diabetes, the complications may be arranged in decreasing order of possible mitigation based on interventions of attaining clinical outcomes. From Tables 1 and 2 the complications may be arranged beginning with Diabetic Retinopathy, Diabetic Neuropathy, Ketoacidosis, Diabetic Nephropathy, Infections/Advanced Infections, Stroke, CAD, Amputations/Gangrene, End-State Renal Disease, Kidney Transplant. From this list of complications, a list of desired clinical outcomes which may assist in promoting the avoidance of these complications and therefore the avoidance of the costs (direct and indirect) associated with the complications is compiled. Based on the desired clinical outcomes to be achieved a list of targeted interventions is generated and provided as a hierarchy of interventions, as described previously. Thus, the patient may be provided with an individualized plan which provides information about the complications which may be expected during the progression of the disease and desired clinical outcomes to be achieved through implementation of various targeted interventions which assist in mitigating the development of such complications. It is to be understood that the list of targeted interventions which the patient is provided are behavioral interventions needed to assist in the achievement of the clinical outcomes. Thus, the targeted interventions are behavioral influences in that the patient is shown what specific behaviors may promote the achievement of desired clinical outcomes which promote a successful strategy for dealing with the complication. The successful strategy includes the avoidance of the complication and/or treatment of the complication.

In the exemplary embodiments, the methods disclosed may be implemented as sets of instructions or software readable by a device (computing system). Further, it is understood that the specific order or hierarchy of steps in the methods disclosed are examples of exemplary approaches. Based upon design preferences, it is understood that the specific order or hierarchy of steps in the method can be rearranged while remaining within the scope and spirit of the present invention. The accompanying method claims present elements of the various steps in a sample order, and are not necessarily meant to be limited to the specific order or hierarchy presented.

It is believed that the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

### REFERENCES

1. August, P. Initial Treatment of Hypertension, N England J Med, Vol. 348, (2003), pp. 610-7.

2. National Institutes of Health. (1997). The Sixth Report of the Joint National Committee on Prevention, Detection, Evaluation, and Treatment of High Blood Pressure, National Institutes of Health (NIH Publication No. 98-4080). Washington, D.C.: National Heart, Lung and Blood Institute.

3. NIH, 2001 Third Report of the National Cholesterol Education Program (NCEP) Expert Panel on Detection, Evaluation and Treatment of High Blood Cholesterol in Adults (Adult Treatment Panel III). Washington, D.C.: National Heart, Lung, and Blood Institute.

4. Actual blood pressure control: are we doing things right?, J Hypertens Suppl, Vol. 16(1), (January 1998), pp. S45-51.

5. HCUP. National Statistics—outcomes for 109 (Acute cerbvrovascular disease) [online], 2000 [retrieved on 2002-12-13]. Retrieved from the Internet: URL: http://hcup.ahrq.gov/HCUPnet.asp.

6. HCUP. National Statistics—outcomes for 101 (Coronary Atherosclerosis) [online], 2000 [retrieved on 2002-12-13]. Retrieved from the Internet: URL: http://hcup.ahrq.gov/HCUPnet.asp.

7. Stroke:Hope Through Research, (retrieved from “The Stroke/Brain Attack Reporter's Handbook,” National Stroke Association. Englewood, Colo.,), National Institute of Neurological Disorder and Stroke (1999), Stroke: Hope Through Research. [online], [retrieved on 2002-12-16]. Retrieved from the Internet: URL: http://www.ninds.nih.gov/health_and_medical/pups/stroke_hope_through_research.htm.

8. Carson, P., T. Giles, M. Higginbotham, N. Hollenberg, W. Kannel, H. Siragy, Angiotensin Receptor Blockers: Evidence for Preserving Target Organs. Clincal Cardiology, Vol. 24, (March 2001), pp. 183-190.

9. Kokkinos P F, Narayan P, Papademetriou V., Exercise as hypertension therapy. Cardiol Clin, Vol. 19(3), (August 20010, pp. 507-16.

10. Haennel R G, Lemire F., Physical activity to prevent cardiovascular disease. How much is enough? Can Fam Physician, Vol. 48, (January 2002), pp. 65-71.

11. Fagard R H., Prescription and results of physical activity. J Cardiovasc Pharmocol, Vol. 25, Suppl 1, (1995), pp. S20-7.

12. Special Populations: Stroke-2002 Edition [online], 2002 [retrieved on 2002-12-31]. Retrieved from the Internet: URL: http://professional.asha.org/resources/factsheet/stroke.cfm.

13. Farmingham study. New England Journal of Medicine [online], Nov. 1, 2001, [retrieved on 2002-12-16]. Retrieved from the Internet: URL: http://www.nhlbi.nih.gov/new/press/01-10-31.htm.

14. High blood pressure statistics. American Heart Association [online], [retrieved on 2002-12-10]. Retrieved from the Internet: URL: http://www.americanheartassociation.org.

15. Uehara Y, Arakawa K., Non pharmacological therapy in hypertensive patients—effect of physical exercise on hypertension. Nippon Rinsho, Vol. 55(8), (August 1997), pp. 2034-8.

16. American Heart Association. Acc/AHA/ACP-ASIM. Guidelines for the Management of patients with chronic Stable angina, 1999, (4S Trial) [online]. [Retrieved on 2002-12-9]. Retrieved from the Internet: URL: http:216.185.112.5/presenter.jhtml?identifier=2113.

17. Primary prevention of heart disease and stroke: a simplified approach to estimating risk of events and making drug treatment decisions, CMAJ 1997:157:422-8. [Online] [retrieved on 2002-3-19]. Retrieved from the Internet: URL: http://www.cma.ca/cmaj/vol-157/issure-4/0422.htm.

18. National Center for Health Statistics. Health, United States, 2002 with Chartbook on Trends in the Health of Americans. Hyattsville, Md. [Online] [retrieved on 2002]. Retrieved from the Internet: URL: http://www.cdc.gov/nchs/products/pubs/pubd/hus/o2husdes.htm.

19. Diabetes Dateline, National Diabetes Information Clearinghouse, Diet and Exercise Delay Diabetes and Normalize Blood Glucose. NIDDk, (NIH), Spring 2002, National Institutes of Diabetes and Digestive and Kidney Diseases.

20. American Diabetes Association, Standards of Medical Care for Patients with Diabetes Mellitus. Diabetes Care, Vol 26, (2003), pp. S33-S50. [Online], [retrieved on 2003]. Retrieved from the Internet: URL: http://care.diabetesjournals.org/cgi/content/full/26suppl_{—}1/s33.

21. American Diabetes Association, Hyperglycemic Crisis in Patients with Diabetes Mellitus, 2003, Diabetes Care Vol. 26, (2003), pp. S109-S117.

22. Fonseca, V., M. Munshi, L. Merin, J. Bradford. Diabetic Retinopathy: A Review for the Primary Care Physician. [Online], September 1996, [retrieved on 2003-1-15]. Retrieved from the Internet: URL: http:www.sam.org/smj/96sept1.htm.

23. [Online], Diabetic neuropathy, Health>Encyclopedia>Diabetic neuropathy. Retrieved from the Internet: URL: http://health.yahoo.com/health/dc/000693/0.html.

24. Brammer, L., K. Fukuda, A. Klimov, N. Cox, Influenza VPD Surveillance Manual, Influenza 3rd edition, Vol. 5-1, (2002), Chapter 5.

25. New England Journal of Medicine, Vol. 329(14), (Sep. 30, 1993). National Diabetes Information Clearinghouse, 1 Information Way, Bethesda, Md. 20892-3560. NIH Publication No. 02-3874 October 2001.

26. Precipating factors for diabetic ketoacidosis. Saudi Med J., Vol. 23(2), (February 2002), pp. 173-6.

27. Health Care and Economic Costs of Diabetes (National Diabetes Fact Sheet). The Centers for Disease Control and Prevention of the U.S. Department of Health and Human Services [online], Nov. 1, 1998. Retrieved from the Internet: URL: http://www.advamed.org/medtechsaving/diabetestreatment.html.

28. [Online], [retrieved on 2003-2-10]. Retrieved from the Internet: URL: http://www.pancreatstonic.com/facts_figures/overview.htm.

29. Health Insurance rate for 2003, Conference News [online]. Retrieved from the Internet: URL: http://www.neumc.org/Financial/health-Insurance-2003.htm.

30. New State Data show obesity and Diabetes Still on the Rise. Center for Disease Control and Prevention [online]. Retrieved from the Internet: URL: http:www.cdc.gov/od/oc/media/pressrel/r021231.htm.

31. Diabetic ketoacidosis. Browse Guides/Internal Medicine/Endocrine and Metabolic disorders [online]. [Retrieved on 2003-2-21]. Retrieved from the Internet: URL: http:www.eboncall.co.uk/jsp/guide/dka/dka_prevalence_{—}1.htm.

32. The Kidney and CVD, An overview of the Kidney Cardiovascular Disease (CVD). American Heart Association [online]. Retrieved from the Internet: URL: http://www.americanheart.org/presenter.jhtml?identifier=681.

33. Kidney and Urologic Disease Statistics for the United States. NIDDK-National Kidney and Urologic Diseases Information Clearinghouse [online]. Retrieved from the Internet: URL: http://www.niddk.nih.gov/health/kidney/pubs/kustats.htm.

34. The incidence of Retinopathy 10 years after diagnosis in young Adult people with diabetes: Results from the nationwide population-based diabetes incidence study in Sweden (DISS). Diabetes Care, Vol. 26(2), (February 2003), pp. 349-354.

35. Evaluating the management of diabetic retinopathy in a teaching center. Eur J Ophthalmol, Vol. 12(6), (November-December 2002), pp. 488-94.

36. Blood pressure, lipids, and obesity are asscociated with retinopahty: the hoorn study. Diabetes Care, Vol 25(8), (August 2002), pp. 1320-5.

37. [Retrieved on 2003-2-10]. Retrieved from the Internet: URL: http://www.pancreatstonic.com/facts_figures/overview.htm.

38. Prevelance of Diabetic nephropathy [online]. Retrieved from the Internet: URL: http://www.intmed.mcw.edu/gimcem/nephropathy/s1d006.htm.

39. Kidney Disease of Diabetes. NIDDK [online], [retrieved on 2003-2-10]. Retrieved from the Internet: URL: http://www.niddk.nih.gov/health/kidney/pubs/kdd/kdd.htm.

40. United States Renal Data System, Annual Data Report. URSDS Coordinating Center [online], [retrieved on 2002]. Retrieved from the Internet: URL: http://www.usrds.org/adr.htm.

41. Edward J. Boyko, Benjamin A. Lipsky. National Health Interview Survey, Infections and Diabetes, (1989), Chapter 22.

42. PMS Evans, C Williams, M D Page, J C Alcolado, C A Abbott, E R E van Ross, J. Kulkanrni, J. E Shaw, A L Carrington, and A J M Boulton, Laterality of lower limb amputation in diabetic patients, BMJ, Vol. 319(7205), (Jul. 31, 1999), pp. 318-318.

43. Richard M Stillman, Diabetic Ulcers, 1/25/02, Chief of staff, Department of Surgery, Northwest Medical Center [online], Jan. 25, 2002, [retrieved on 2003-2-19]. Retrieved from the Internet: URL: http://www.emedicine.com/MED/topic551.htm.

44. Russell M W, Huse D M, Drowns S, Hamel E C, Hartz S C. Direct Medical costs of coronary artery disease in the United States, AM J Caridiol., Vol 81(9), (May 1998), pp. 1110-5.

45. (Abstract). [Online] [retrieved on 2002-12-15]. Retrieved from the Internet: URL: http://www.ncbi.gov/.../query.fcgi?cmd=retrieve&db=PubMed&list_uids=9605051& dopt=Abstract.

46. American Stroke Association, American Heart Association. Heart Disease and Stroke Statistics-2003 Update. Drugs, Vol 62(9), (2002), pp. 1381-1405, [online], [retrieved on 2003]. Retrieved from the Internet: URL: http://www.americanheart.org/presenter.jhtml?identifier=1864.

47. American Heart Association Guidelines-preventative treatment of cardiac conditions www.Amercianheart.org exp-1) American Heart Association, Guidelines—ACC/AHA/ACP-ASIM Guidelines for the Management of Patients with Chronic Stable Agina—Part IVD, [online], [retrieved on 2003-2-24]. Retrieved from the Internet: URL: http://216.185.112.5/presenter.jhtml?identifier=2113, and 2) 1999 Update:ACC/AHA Guidelines for the Managment of Patients with acute myocardial infarction: Executive Summary and Recommendations, Circulation, 1999;100:1016-1030.

48. Heart Transplant Statistics, AHA, American Heart Association. 2002 Heart and Stroke Statistical Update. Dallas, Tex.: American Heart Association, 2001.

49. American Association of Clinical Endocrinologists Diabetes Guidelines, Vol. 6 No. 1, January/February 2000.

50. G. Druss, et al., The Most Expensive Medical Conditions in America, Health Affairs, Vol. 21, No. 4, July-August 2002.

51. Low Back Pain Fact Sheet. American Academy of Orthopaedic Surgeons [online], [retrieved on 2003-9-11]. Retrieved from the Internet: URL: http//www.aaos.org/wodhtml/wash/rhd_lowbackpain.htm.

52. Back Pain-Fact Sheet. American Academy of Orthopedic Surgeons [online], [retrieved on 2003-5-19]. Retrieved from the Internet: URL: www.aaos.org, downloaded 5/19/03.

53. Institute for Clinical Systems Improvement (ICSI). Adult Low Back Pain. Bloomington (Minn.): Institute for Clinical Systems Improvement (ICSI); 2002 September 61p.

54. U.S. Census Bureau, Census 2000 Redistricting Data (Public Law 94-171) Summary File, tables PL1 and PL3

55. Sneider, J., Racine health care alliance picks Wausau over Blue Cross. The Business Journal Serving Greater Milwaukee, week of Jan. 27, 1997 print edition and [online], [retrieved on 2003-2-14]. Retrieved from the Internet: URL: http://milwaukee.bizjournal.com/milwaukee/stories/1997/01/27/story3.html.

56. Crutsinger, Marin, Low inflation rate caps best run in 30 years. South Coast Today [online], [retrieved on 2003-2-14]. Retrieved from the Internet: URL: http://www.s-t.com/daily/02-96/02-02-97/07a03910.htm.

57. Hoover, Kent. Federal Government's Annual report on national health expenditures.

Rising costs overshadow rights of patients in health care debate. Houston Business Journal, Jan. 18, 2002 print edition Washington Beat and [online], [retrieved on 2002-1-21].

58. Myers, D., and D Peak. Health Insurance Rate for 2003. Conference News [online], Oct. 23, 2002, [retrieved on 2003-2-10]. Retrieved from the Internet: URL: http://www.neumc.org/Financial/Health-Insurance-2003.htm.

59. Abbott, R. K., Health Care Costs:No Easy Answers. Journal of Compensation and Benefits, Vol. 18, No.3 (May-June 2002), pp 31-36, [online], [retrieved on 2003-2-19].

60. Weiss et al., J. Allergy Clin. Immunol., Vol 107, (2001), pp. 3-8.

61. CIA World Factbook [online], 1998. Retrieved from the Internet: URL: www.greekorthodoxchurch.org/wfb1998/united_states/united_states_people.html.

62. Basic Facts about Asthma. Center for Disease Control and Prevention [online], [retrieved on 2003-12-4]. Retrieved from the Internet: URL: www.cdc.gov/nech/airpollution/asthma/faqs.htm.

What is claimed is:

1. A method of projecting health care cost savings for a complication associated with a disease, comprising:
determining an indirect cost associated with the complication;
determining a mitigating factor associated with complication; and
calculating a projected indirect cost savings based on the indirect cost and mitigating factor.

2. The method of claim 1, wherein the mitigating factor is a percent mitigable associated with the complication of the disease.

3. The method of claim 1, further comprising the steps of determining a direct cost associated with the complication.

4. The method of claim 1, further comprising the step of calculating a total cost savings based on the indirect cost, direct cost and mitigating factor.

5. The method of claim 1, further comprising the step of determining a second mitigating factor for the direct costs.

6. The method of claim 1, wherein the disease may be selected from the group consisting of diabetes, asthma, back pain, coronary artery disease, or chronic (congestive) heart failure.

7. The method of claim 1, wherein the method may be implemented as a computer readable set of instructions executable upon a computing system.

8. A method of projecting health care cost savings for a complication associated with a disease, comprising:
determining an indirect cost associated with the complication;
determining a direct cost associated with the complication;
determining a mitigating factor associated with the indirect and direct costs;
calculating a projected total cost savings based on the indirect cost, direct cost and mitigating factor.

9. The method of claim 8, further comprising the step of determining a second mitigating factor for the direct costs.

10. The method of claim 8, wherein the disease may be selected from the group consisting of diabetes, asthma, back pain, coronary artery disease, or chronic (congestive) heart failure.

11. The method of claim 8, wherein the mitigating factor is a percent mitigable associated with the complication of the disease.

12. The method of claim 8, wherein the mitigating factor is based on attainment of a desired clinical outcome selected from the group consisting of a HbA1c level, blood pressure, cholesterol level.

13. The method of claim 12, wherein the attainment of a desired clinical outcome is through execution of a targeted intervention selected from the group consisting of medication management, diet, exercise, periodicity or smoking cessation.

14. The method of claim 8, wherein the method may be implemented as a computer readable set of instructions executable upon a computing system.

15. A computer readable set of instructions allowing for the projection of health care cost savings for a complication associated with a disease, comprising:
a first instruction determines a direct cost savings associated with at least one complication;
a second instruction determines an indirect cost savings associated with the at lest one complication; and
a third instruction for the calculation of a projected total cost savings based on the direct and indirect costs associated with the complication.

16. The computer readable set of instructions of claim 15, further comprising an instruction allowing for the identification of the disease.

17. The computer readable set of instructions of claim 15, further comprising an instruction for constructing a list of a plurality of complications associated with the identified disease.

18. The computer readable set of instructions of claim 15, further comprising an instruction for calculating only one of a projected direct cost savings or a projected indirect cost savings.

19. The computer readable set of instructions of claim 15, wherein the projected total cost savings may include projected direct and indirect costs for two or more complications.

20. The computer readable set of instructions of claim 15, further comprising an instruction for indicating specific interventions for the attainment of a desired clinical outcome.

21. A method for providing a patient care program, comprising:
identifying a medical condition of the patient;
identifying a complication associated with the progression of the medical condition;
identifying and grouping a clinical outcome to the complication, the clinical outcome having an impact upon the complication;
identifying a targeted intervention for attaining the clinical outcome;
determining the direct and indirect projected cost savings which may be realized based on the complication;
wherein the patient care program is determined from the identification and determination of the disease, the complication and the projected cost savings, and the targeted interventions for achieving the desired clinical outcome.

22. The method of claim 21, wherein the identified medical condition may be selected from the group consisting of diabetes, asthma, back pain, coronary artery disease, or chronic (congestive) heart failure.

23. The method of claim 21, wherein the disease is a plurality of medical conditions.

24. The method of claim 21, wherein the complication is a plurality of complications.

25. The method of claim 21, wherein the clinical outcome is a plurality of clinical outcomes.

26. The method of claim 21, wherein the targeted intervention is a plurality of targeted interventions.
